(19) United States
(12) Patent Application Publication
(10) Pub. No.: US 2021/0299152 A1
(43) Pub. Date: Sep. 30, 2021

(54) ORAL COMPOSITIONS AND METHODS FOR AFFECTING MAMMALIAN OFFSPRING

(71) Applicant: Kalmarna Limited, Tortola, British Virgin Islands (GB)

(72) Inventors: Alon Rosenberg, Rehovot (IL); Abraham Milstein, Rehovot (IL); Ava Marie Firth, Nottinghamshire (GB); Simon Van Dalsem, Alkmaar (NL); Arie Halpern, Pully (CH)

(73) Assignee: Kalmarna Limited, Tortola, British Virgin Islands (GB)

(21) Appl. No.: 17/051,644

(22) PCT Filed: Apr. 29, 2019
(86) PCT No.: PCT/IL2019/050471

§ 371 Date: Oct. 29, 2020

Related U.S. Application Data
(60) Provisional application No. 62/664,173, filed on Apr. 29, 2018.

(51) Int. Cl. A61K 31/704; A61K 31/194; A61K 31/198; A61K 9/00; A61K 45/06; A61P 3/02; A23K 20/163; A23K 20/105; A23K 20/142; A23K 50/30

(52) U.S. Cl. CPC A61K 31/704; A61K 31/194; A61K 31/198; A61K 9/0053; A23K 50/30; A61P 3/02; A23K 20/163; A23K 20/105; A23K 20/142; A61K 45/06

(57) ABSTRACT

The present invention discloses methods and compositions for nourishing newly-borne mammals; specifically, reducing antibiotic usage, increasing weight and increasing immunoglobulin concentrations in said newly borne mammals, following oral treatment of pregnant mammals of said newly-borne mammals. The compositions of the current invention have chicken, beef, fish or apple flavouring.

### FIELD OF THE INVENTION

The invention generally pertains to compositions and methods for nourishing of mammalian offspring. Specifically, the invention relates to palatable oral formulations for reducing antibiotic usage, increasing weight and increasing immunoglobulin concentrations in newly born mammals, following oral treatment of their dams (maternal parent).

### BACKGROUND OF THE INVENTION

Piglet pre-weaning mortality is one of the major reproductive components that affects herd productivity in the swine industry.

In modern swine industry, producers expect up to 30 pigs weaned per sow per year. However, in practice, a high proportion of piglet mortality occurs during pre-weaning period. Thus, factors associated with piglet pre-weaning mortality (PWM) are becoming a major concern in swine industry worldwide. The mean piglet pre-weaning mortality rate in commercial swine herds worldwide varies from 10 to 20%. Based on recent literature, the mean piglet pre-weaning mortality rate is 12.9% in Europe, 9.4% in the Philippines, and 12.2% in Thailand. On average, 50-80% of piglet deaths occur during the first week after birth, with the most critical period being the first 72 h of life; see Nuntapaitoon, Morakot, and Padet Tummaruk. “Factors influencing piglet pre-weaning mortality in 47 commercial swine herds in Thailand.” Tropical animal health and production 50.1 (2018): 129-135, incorporated herein as a reference.

Factors influencing piglet pre-weaning mortality under field conditions include sow factors (e.g., breed, parity, nutritional status, farrowing duration, maternal behavior and LS), piglet factors (e.g., Piglet body weight at birth, birth order and sex) and environmental factors (e.g., ambient temperature and stocking density); see Nuntapaitoon, M., Muns, R., & Tummaruk, P. (2018). Newborn traits associated with pre-weaning growth and survival in piglets. Asian-Australasian Journal of Animal Sciences, 31(2), 237-244 incorporated herein as a reference.

Piglet pre-weaning mortality is a serious and important welfare and economic problem of great concern in swine production. Thus, there is unmet need to reduce pre-weaning mortality inter alia in pigs.

### BRIEF DESCRIPTION OF THE FIGURES

FIG. 1 Percentage of piglets treated with antibiotics, C-Control, P120-Sows given 120 mL/day, P500-Sows given 500 mL/day and

FIG. 2 Immunoglobulin concentrations in piglets (mg/mL), C-Control, P120-Sows given 120 mL/day, P500-Sows given 500 mL/day.

### SUMMARY OF THE INVENTION

It is an object of the invention to disclose a method for nourishing a mammalian offspring, characterized by per os administration to a pregnant mammal an effective dose of formulation comprising at least two members of a group consisting of steviol glycoside, citric acid monohydrate, monosodium glutamate and glycine; along the period of time ranging from third trimester of pregnancy to weaning.

It is another object of the invention to disclose a method for increasing the weaning weight of a mammalian offspring, characterized by per os administration to a pregnant mammal an effective dose of formulation comprising at least two members of a group consisting of steviol glycoside, citric acid monohydrate, monosodium glutamate and glycine; along the period time ranging from third trimester of pregnancy to weaning.

It is another object of the invention to disclose a method for increasing immunoglobulin levels in blood of a mammalian offspring, characterized by per os administration to a pregnant mammal an effective dose of formulation comprising at least two members of a group consisting of steviol glycoside, citric acid monohydrate, monosodium glutamate and glycine; along the period time ranging from third trimester of pregnancy to weaning.

It is another object of the invention to disclose a method for reducing number of mammal offspring treated with antibiotics, characterized by per os administration to a pregnant mammal an effective dose of formulation comprising at least two members of a group consisting of steviol glycoside, citric acid monohydrate, monosodium glutamate and glycine; along the period time ranging from thirds trimester of pregnancy to weaning.

It is another object of the invention to disclose a method for reducing dose of antibiotics required to treat a mammalian offspring up to age of weaning, characterized by per os administration to a pregnant mammal an effective dose of formulation comprising at least two members of a group consisting of steviol glycoside, citric acid monohydrate, monosodium glutamate and glycine; along the period time ranging from third trimester of pregnancy to weaning.

It is another object of the invention to disclose a method for increasing average daily gain of a mammalian offspring, characterized by per os administration to a pregnant mammal an effective dose of formulation comprising at least two members of a group consisting of steviol glycoside, citric acid monohydrate, monosodium glutamate and glycine; along the period time ranging from third trimester of pregnancy to weaning.

It is another object of the invention to disclose an oral isotonic compositions characterized by no residual chlorine taste, useful for nourishing a mammalian offspring by administering the same to a pregnant mammal along the period time ranging from third trimester of pregnancy to weaning, said composition comprising at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for nourishing a mammalian offspring by administering the same to a pregnant mammal along the period time ranging from third trimester of pregnancy to weaning, said composition comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an isotonic drink characterized by no residual chlorine taste, useful for nourishing a mammalian offspring by administering the same to a pregnant mammal along the period time ranging from third trimester of pregnancy to weaning, said composition comprising at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink characterized by no residual chlorine taste, useful for nourishing a mammalian offspring by administering the same to a pregnant mammal along the period time ranging from third trimester of pregnancy to weaning, said composition comprising (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' isotonic drink characterized by no residual chlorine taste, useful for nourishing a mammalian offspring by administering the same to a pregnant mammal along the period time ranging from third trimester of pregnancy to weaning, said composition comprising (a) steviol glycoside, citric acid monohydrate, monosodium glutamate, glycine, mixtures and derivatives thereof; and (b) an apple-flavouring agent.

It is another object of the invention to disclose an apple flavoured horses' isotonic drink characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an isotonic taste enhancer characterized by no residual chlorine taste, useful for nourishing a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

It is another object of the invention to disclose an isotonic taste enhancer characterized by no residual chlorine taste, useful for nourishing a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an isotonic apple flavoured horses' taste enhancer characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an isotonic apple flavoured horses' taste enhancer characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an edible powder characterized by no residual chlorine taste, useful for nourishing a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for nourishing a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible powder characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible powder characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an edible gel characterized by no residual chlorine taste, useful for nourishing a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible gel compositions characterized by no residual chlorine taste, useful for nourishing a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible isotonic gel characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible isotonic gel characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose a solid shear thinning thixotropic edible characterized by no residual chlorine taste, useful for nourishing a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures

It is another object of the invention to disclose a solid shear thinning thixotropic edible gel characterized by no residual chlorine taste, useful for nourishing a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' solid shear thinning thixotropic edible gel characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' solid shear thinning thixotropic edible gel characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an oral isotonic compositions characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an isotonic drink characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an apple flavoured horses' isotonic drink characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

It is another object of the invention to disclose an apple flavoured horses' isotonic drink characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an an edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an an edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

It is another object of the invention to disclose an an edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

It is another object of the invention to disclose an an edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

It is another object of the invention to disclose an apple flavoured horses' edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

It is another object of the invention to disclose an apple flavoured horses' edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an oral isotonic compositions characterized by no residual chlorine taste, useful for increasing the weaning weight of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for increasing the weaning weight of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an isotonic drink characterized by no residual chlorine taste, useful for increasing the weaning weight of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' isotonic drink characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) an apple-flavouring agent.

It is another object of the invention to disclose an apple flavoured horses' isotonic drink characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an isotonic taste enhancer characterized by no residual chlorine taste, useful increasing the weaning weight of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

It is another object of the invention to disclose an isotonic taste enhancer characterized by no residual chlorine taste, useful for increasing the weaning weight of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an isotonic apple flavoured horses' taste enhancer characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an isotonic Apple flavoured horses' taste enhancer characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an edible powder characterized by no residual chlorine taste, useful for increasing the weaning weight of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for increasing the weaning weight of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible powder characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible powder characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an edible gel characterized by no residual chlorine taste, useful for increasing the weaning weight of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible gel compositions characterized by no residual chlorine taste, useful for increasing the weaning weight of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible isotonic gel characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible isotonic gel characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose a solid shear thinning thixotropic edible characterized by no residual chlorine taste, useful for increasing the weaning weight of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose a solid shear thinning thixotropic edible gel characterized by no residual chlorine taste, useful for increasing the weaning weight of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' solid shear thinning thixotropic edible gel characterized by no residual chlorine taste, useful for increasing the weaning weight of a mammalian offspring administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' solid shear thinning thixotropic edible gel characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an oral isotonic compositions characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an isotonic drink characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an apple flavoured horses' isotonic drink characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

It is another object of the invention to disclose an apple flavoured horses' isotonic drink characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an edible powder characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

It is another object of the invention to disclose an edible powder characterized by no residual chlorine taste, useful increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

It is another object of the invention to disclose an apple flavoured horses' edible powder characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

It is another object of the invention to disclose an apple flavoured horses' edible powder characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an oral isotonic compositions characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for increasing immunoglobulin levels in blood of a mammalian offspring by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) an apple-flavouring agent.

It is another object of the invention to disclose an apple flavoured horses' isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an isotonic taste enhancer characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

It is another object of the invention to disclose an isotonic taste enhancer characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an isotonic apple flavoured horses' taste enhancer characterized by increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an isotonic apple flavoured horses' taste enhancer characterized by increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an edible powder characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible powder characterized by no residual chlorine taste, useful increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an edible gel characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible gel compositions characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible isotonic gel characterized by increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible isotonic gel characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose a solid shear thinning thixotropic edible characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose a solid shear thinning thixotropic edible gel characterized by no residual chlorine taste, useful for increasing immunoglobulin levels in blood of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' solid shear thinning thixotropic edible gel characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' solid shear thinning thixotropic edible gel characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an oral isotonic compositions characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an apple flavoured horses' isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

It is another object of the invention to disclose an apple flavoured horses' isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

It is another object of the invention to disclose an edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

It is another object of the invention to disclose an apple flavoured horses' edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

It is another object of the invention to disclose an apple flavoured horses' edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an oral isotonic compositions characterized by no residual chlorine taste, useful for reducing number of mammal offspring treated with antibiotics by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for reducing number of mammal offspring treated with antibiotics by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an isotonic drink characterized by no residual chlorine taste, useful for reducing number of mammal offspring treated with antibiotics by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' isotonic drink characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) an apple-flavouring agent.

It is another object of the invention to disclose an apple flavoured horses' isotonic drink characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an isotonic taste enhancer characterized by no residual chlorine taste, useful for reducing number of mammal offspring treated with antibiotics by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

It is another object of the invention to disclose an isotonic taste enhancer characterized by no residual chlorine taste, useful for reducing number of mammal offspring treated with antibiotics by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an isotonic apple flavoured horses' taste enhancer characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an isotonic Apple flavoured horses' taste enhancer characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an edible powder characterized by no residual chlorine taste, useful for reducing number of mammal offspring treated with antibiotics by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for reducing number of mammal offspring treated with antibiotics by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible powder characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible powder characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an edible gel characterized by no residual chlorine taste, useful for reducing number of mammal offspring treated with antibiotics by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible gel compositions characterized by no residual chlorine taste, useful for reducing number of mammal offspring treated with antibiotics by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible isotonic gel characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible isotonic gel characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an asolid shear thinning thixotropic edible characterized by no residual chlorine taste, useful for reducing number of mammal offspring treated with antibiotics by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose ana solid shear thinning thixotropic edible gel characterized by no residual chlorine taste, useful reducing number of mammal offspring treated with antibiotics by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' solid shear thinning thixotropic edible gel characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' solid shear thinning thixotropic edible gel characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an oral isotonic compositions characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an isotonic drink characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an apple flavoured horses' isotonic drink characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

It is another object of the invention to disclose an apple flavoured horses' isotonic drink characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

It is another object of the invention to disclose an edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

It is another object of the invention to disclose an apple flavoured horses' edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

It is another object of the invention to disclose an apple flavoured horses' edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an oral isotonic compositions characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) an apple-flavouring agent.

It is another object of the invention to disclose an apple flavoured horses' isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an isotonic taste enhancer characterized by no residual chlorine taste, useful reducing dose of antibiotics required to treat a mammalian offspring up to age of weaning by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

It is another object of the invention to disclose an isotonic taste enhancer characterized by no residual chlorine taste, useful reducing dose of antibiotics required to treat a mammalian offspring up to age of weaning by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an isotonic apple flavoured horses' taste enhancer characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an isotonic Apple flavoured horses' taste enhancer characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an an edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an edible gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible gel compositions characterized by no residual chlorine taste, useful reducing dose of antibiotics required to treat a mammalian offspring up to age of weaning by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible isotonic gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible isotonic gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose ana solid shear thinning thixotropic edible characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose ana solid shear thinning thixotropic edible gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' solid shear thinning thixotropic edible gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' solid shear thinning thixotropic edible gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an oral isotonic compositions characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an apple flavoured horses' isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

It is another object of the invention to disclose an apple flavoured horses' isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

It is another object of the invention to disclose an edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

It is another object of the invention to disclose an apple flavoured horses' edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

It is another object of the invention to disclose an apple flavoured horses' edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an oral isotonic compositions characterized by no residual chlorine taste, useful for increasing average daily gain of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for increasing average daily gain of a mammalian offspring by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an isotonic drink characterized by no residual chlorine taste, useful for increasing average daily gain of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' isotonic drink characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) an apple-flavouring agent.

It is another object of the invention to disclose an apple flavoured horses' isotonic drink characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an isotonic taste enhancer characterized by no residual chlorine taste, useful for increasing average daily gain of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

It is another object of the invention to disclose an isotonic taste enhancer characterized by no residual chlorine taste, useful for increasing average daily gain of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an isotonic apple flavoured horses' taste enhancer characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an isotonic Apple flavoured horses' taste enhancer characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is another object of the invention to disclose an edible powder characterized by no residual chlorine taste, useful for increasing average daily gain of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for increasing average daily gain of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible powder characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible powder characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an edible gel characterized by no residual chlorine taste, useful for increasing average daily gain of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose an edible gel compositions characterized by no residual chlorine taste, useful for increasing average daily gain of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible isotonic gel characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' edible isotonic gel characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose a solid shear thinning thixotropic edible characterized by no residual chlorine taste, useful for increasing average daily gain of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is another object of the invention to disclose a solid shear thinning thixotropic edible gel characterized by no residual chlorine taste, useful for increasing average daily gain of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' solid shear thinning thixotropic edible gel characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is another object of the invention to disclose an apple flavoured horses' solid shear thinning thixotropic edible gel characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

It is another object of the invention to disclose an oral isotonic compositions characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an isotonic drink characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an apple flavoured horses' isotonic drink characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

It is another object of the invention to disclose an apple flavoured horses' isotonic drink characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an edible powder characterized by no residual chlorine taste, useful increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

It is another object of the invention to disclose an edible powder characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

It is another object of the invention to disclose an edible powder characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

It is still another object of the invention to disclose an apple flavoured horses' edible powder characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

It is also another object of the invention to disclose an apple flavoured horses' edible powder characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is also another object of the invention to disclose an oral isotonic compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose an oral isotonic compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

It is also another object of the invention to disclose an isotonic drink characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

It is also another object of the invention to disclose an isotonic drink characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

It is also another object of the invention to disclose an isotonic taste enhancer characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

It is also another object of the invention to disclose an isotonic taste enhancer characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is also another object of the invention to disclose an isotonic apple flavoured horses' taste enhancer characterized by chicken flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is also another object of the invention to disclose an isotonic Apple flavoured horses' taste enhancer characterized by chicken flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is also another object of the invention to disclose an edible powder characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose an edible powder characterized by chicken flavour and no residual chlorine taste, useful increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is also another object of the invention to disclose an edible gel characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose an edible gel compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is also another object of the invention to disclose a solid shear thinning thixotropic edible characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose a solid shear thinning thixotropic edible gel characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

It is also another object of the invention to disclose an oral isotonic compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is also another object of the invention to disclose an oral isotonic compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

It is also another object of the invention to disclose an isotonic drink characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

It is also another object of the invention to disclose an isotonic drink compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

It is also another object of the invention to disclose an edible powder characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is also another object of the invention to disclose an edible powder characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

It is also another object of the invention to disclose an edible powder characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

It is also another object of the invention to disclose an edible powder characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

It is also another object of the invention to disclose an oral isotonic compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose an oral isotonic compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

It is also another object of the invention to disclose an isotonic drink characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

It is also another object of the invention to disclose an isotonic drink characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

It is also another object of the invention to disclose an isotonic taste enhancer characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

It is also another object of the invention to disclose an isotonic taste enhancer characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is also another object of the invention to disclose an isotonic apple flavoured horses' taste enhancer characterized by fish flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is also another object of the invention to disclose an isotonic Apple flavoured horses' taste enhancer characterized by fish flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is also another object of the invention to disclose an edible powder characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose an edible powder characterized by fish flavour and no residual chlorine taste, useful increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is also another object of the invention to disclose an edible gel characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose an edible gel compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is also another object of the invention to disclose a solid shear thinning thixotropic edible characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose a solid shear thinning thixotropic edible gel characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

It is also another object of the invention to disclose an oral isotonic compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is also another object of the invention to disclose an oral isotonic compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

It is also another object of the invention to disclose an isotonic drink characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

It is also another object of the invention to disclose an isotonic drink compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

It is also another object of the invention to disclose an edible powder characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is also another object of the invention to disclose an edible powder characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

It is also another object of the invention to disclose an edible powder characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

It is also another object of the invention to disclose an edible powder characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

It is also another object of the invention to disclose an oral isotonic compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose an oral isotonic compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

It is also another object of the invention to disclose an isotonic drink characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

It is also another object of the invention to disclose an isotonic drink characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

It is also another object of the invention to disclose an isotonic taste enhancer characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

It is also another object of the invention to disclose an isotonic taste enhancer characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is also another object of the invention to disclose an isotonic apple flavoured horses' taste enhancer characterized by beef flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

It is also another object of the invention to disclose an isotonic Apple flavoured horses' taste enhancer characterized by beef flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

It is also another object of the invention to disclose an edible powder characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose an edible powder characterized by beef flavour and no residual chlorine taste, useful increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is also another object of the invention to disclose an edible gel characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose an edible gel compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

It is also another object of the invention to disclose a solid shear thinning thixotropic edible characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

It is also another object of the invention to disclose a solid shear thinning thixotropic edible gel characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

It is also another object of the invention to disclose an oral isotonic compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is also another object of the invention to disclose an oral isotonic compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

It is also another object of the invention to disclose an isotonic drink characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

It is also another object of the invention to disclose an isotonic drink compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

It is also another object of the invention to disclose an edible powder characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is also another object of the invention to disclose an edible powder characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

It is also another object of the invention to disclose an edible powder characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

It is also another object of the invention to disclose an edible powder characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, is adapted to remain apparent to those skilled in the art, since the generic principles of the present invention have been defined specifically to provide compositions and methods for nourishing newly-borne mammals. Specifically, the present invention discloses method and composition for reducing antibiotic usage, increasing weight and increasing immunoglobulin concentrations in said newly borne mammals, following oral treatment of pregnant mammals of said newly-borne mammals.

Newborn piglets are highly dependent on colostrum to use it as energy substrate for thermoregulation and growth, and also to acquire passive immunity crucial for their future survival;

Sows' nutrition will influence foetal development and piglet birth weight, and is determinant to ensure a proper colostrum/milk production. Finally, ambient temperature has an important impact on piglet survival because piglets are very sensitive to cold stress. The housing system used in the farrowing room seems to influence the incidence of crushing. Promising results have been obtained using recent designs that combine initial confinement of the sow with the subsequent ability to move within the same pen. Different management strategies to deal with PWM are usually performed by producers around farrowing. However, there is a lack of scientific evidence on techniques, such as oral supplementation of piglets, cross-fostering, nurse sow systems, or artificial rearing of piglets, and further research should be of interest.

As used herein after, the term “pet” or “a companion animal” generally refers hereinafter to an animal kept primarily for a person's company, protection, or entertainment rather than as a working animal, livestock, or laboratory animal. Popular pets are often noted for their attractive appearances, intelligence, and relatable personalities. Two of the most popular pets are dogs and cats. Other animals commonly kept include: pigs, ferrets, rabbits; rodents, such as gerbils, hamsters, chinchillas, rats, and guinea pigs; avian pets, such as parrots, passerines, and fowl; reptile pets, such as turtles, lizards and snakes; aquatic pets, such as fish, freshwater and saltwater snails, and frogs; and arthropod pets, such as tarantulas and hermit crabs. Small pets may be grouped together as pocket pets, while the equine species is generally considered the largest companion animal.

The compositions of the current invention have chicken, beef, fish or apple flavouring.

The term ‘apple-flavoring agent’ refers herein after, inter alia and in a non-limiting manner, to one or more of the following: apple extracts, apple flavor No. 64625, malic acids, esters, salts and derivatives thereof, apple flavor fragrance, e.g., as defined in CN patent applications 107279615, 104830607 and 105212170, JP patent application 2011152095, Maxarome AC 1632, all are incorporated herein as references, taste enhancers, emulsifiers and stabilizers thereof.

Maxarome AC 1632 is a concentrated, high performance flavour developed specifically for inclusion into animal feeds where it will improve palatability and encourage feed intake. Maxarome AC 1632 includes Vanillin and other aldehydes, together with mixed esters. Maxarome AC 1632 has the following characteristics: Physical Appearance: Beige-pale brown free flowing powder; Volatile Matter: 38.94%; Solubility: Insoluble; Bulk Density: 0.625 g/cm3; Flavour Classification: Nature Identical; Flavour Type: Spicy Apple; Product Classification: Conforms to EU Regulation 1831/2003—Annex 1, 2(b); Pre-mixture of flavouring compounds; Added Colouring: None Added; Antioxidants: None Added.

As used herein after, the term “microbiome” generally refers hereinafter to symbiotic microorganisms evolutionarily adapted to life in relatively open human organs, primarily bacteria in the gut and also body niches on the basis of mutually beneficial relations. The microbiome comprises several hundred different species of microorganisms, including archaea, viruses and fungi, although anaerobic bacteria is the most studied group since they are the most abundant These microbial communities are acquired at birth and are essential for maintaining body homeostasis. The interaction between the host and the microbiome is dynamic and controlled by a vast number of genetic and environmental factors, such as age, geography, alcohol or drug intake and diet.

Prebiotics are food ingredients that induce the growth or activity of beneficial microorganisms (e.g., bacteria and fungi). The most common example is in the gastrointestinal tract, where prebiotics can alter the composition of organisms in the gut microbiome. The term ‘prebiotic’ hence refers hereinafter, inter alia and in a non-limiting manner, to both of endogenous and exogenous sources-driven prebiotics, and to at least one member of a group consisting of edibles being non-digestible and resistant to breakdown by stomach acid and enzymes in the human gastrointestinal tract; edibles selectively fermented by intestinal microorganisms; and edibles selectively target and stimulate the growth and activity of beneficial bacteria; see Hutkins R W et al., (2016). “Prebiotics: why definitions matter”. Curr Opin Biotechnol. 37: 1-7. The term also refers to fibers that are non-digestible food ingredients and beneficially affect the host's health by selectively stimulating the growth and/or activity of some genera of microorganisms in the colon, e.g., lactobacilli and bifidobacteria; and non-viable food component that confer health benefit(s) on the host associated with modulation of the microbiota. The term also refers to synbiotics, e.g., food ingredients or dietary supplements combining probiotics and prebiotics in a form of synergism, see DeVrese M, Schrezenmeir J (2008) Probiotics, prebiotics, and synbiotics in food biotechnology (pp. 1-66). Springer Berlin Heidelberg which is incorporated herein as a reference.

It is in the scope of the invention wherein aforesaid non-genetically-modified compositions, products and formulations comprises or based on commercially available Px (trade mark) by Tonisity International Ltd, namely, a composition, e.g., defined below:

| | (%) |
|---|---|
| glucose anhydrous | 1.5540 |
| monosodium glutamate | 0.3900 |
| sodium chloride, vacuum dried | 0.2201 |
| citric acid anhydrous | 0.1860 |
| potassium chloride | 0.1500 |
| glycine | 0.1500 |
| hydrolyzed whey protein isolate | 0.1500 |
| monosodium dihydrogen phosphate | 0.1000 |
| xanthan gum | 0.0500 |
| 1-glutamic acid | 0.0400 |
| stevia (min. 85% steviol glycoside extract) | 0.0100 |
| ingredients | 3.0000 |

demineralized water

The composition consists at least two members of a group consisting of steviol glycoside, citric acid monohydrate, monosodium glutamate and glycine. Additionally or alternatively, this composition characterized by that that the composition consists at least three members of a group consisting of steviol glycoside, citric acid monohydrate, monosodium glutamate and glycine. Additionally or alternatively, this composition is characterized by that that said composition consisting of steviol glycoside, citric acid monohydrate, monosodium glutamate and glycine.

### Example 1

Setting: A study using Px was conducted in a sow farm located in Spain, during July and August 2017. The farm had experienced a PRRS outbreak in the previous month and had chronic issues with neonatal rotavirus diarrhea. The average daily temperature in the area during this period was 30° C. (86° F.), with maximum temperatures reaching 35° C. (95° F.). The farrowing rooms were not air-conditioned. The historic average of live-born pigs was 14.7 pigs/litter, but in the month previous to this study the average live-born was only 12 pigs/litter due to the PRRS outbreak. The average number of live-born pigs was 11.7 pigs/litter in this study. Treatments: 103 sows (Tai Zumu×Landrace×Large White) and their litters (1208 piglets) were enrolled in the study and randomized to one of three groups, which were Control (C), sows receiving 120 mL of Px (P120) or sows receiving 500 mL of Px (P500). Sows receiving Px were given either 120 mL or 500 mL of 3% Px solution once daily for 7 days pre-farrowing and once daily for 7 days pre-weaning. Sows were fed according to the farm's standard feed curve three times daily, and were given the Px solution as top-dressing on their second feeding on treatment days.

All litters in the 3 treatment groups were given 500 mL of Px/litter/day in an open pan, once daily from day 2-8 of age. All sows and piglets had access to an automatic drinker. Creep feed was offered to all litters beginning at 10 days of age.

The usual farm treatment for neonatal diarrhea consisted of a combination injection of amoxicillin and gentamicin. Any treatments were administered by barn staff according to standard operating protocols and were not controlled by the study protocol.

Data collection: At entry to the farrowing house, individual sow weight and back fat was measured. Each sow's feed intake was measured daily, and samples of feed were taken daily to allow calculation of dry matter intake throughout lactation. Sow weight and back fat was measured again on the day of weaning.

Within 24 h of birth, piglets were individually ear-tagged and weighed. Blood samples for serum immunoglobulin assay were taken from 20 piglets from primiparous sows in each treatment group at 24 h of age. Piglets were again weighed at 6 days before weaning, and at weaning (˜21 days of age). Average daily gain (ADG) at weaning was calculated based on actual days of age. Piglet mortalities (with probable cause of death and corpse weight) were recorded daily. Fecal scores per litter and individual antibiotic injections given to either sows or piglets were recorded daily.

Statistical Analysis: Statistical analysis of sow-related variables including body weight and back fat was performed using a generalized linear model with the sow as the experimental unit, using parity class (primiparous, multiparous) and treatment group as co-factors. Statistical analysis for piglet body weight, ADG, creep feed intake, fecal score and survival rate was performed using a mixed model with the sow (litter) as the experimental unit and including sow parity class as a co-factor. The number of piglets receiving one or more antibiotic injections (Amoxicillin and Gentamicin) per treatment (Control, P120 and P500) and parity class were tabulated in contingency tables and the probabilities were calculated using Fisher's exact test. Odds ratios were also calculated for antibiotic usage in piglets using a chi-square test. All statistical analysis was performed using SAS software (version 9.4).

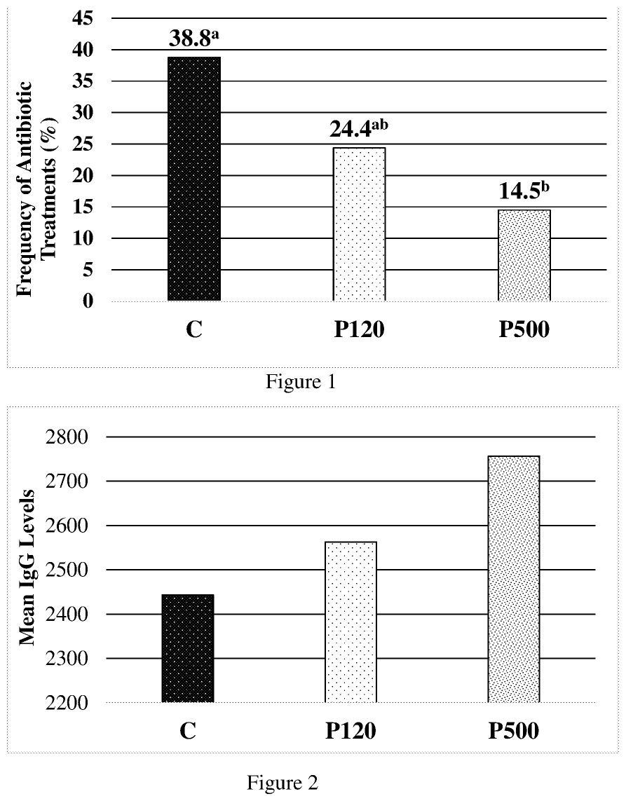

Results: Antibiotic usage: Results of the study showed statistically significant (P<0.01) differences in the number and percentage of pigs in each group that were given antibiotic injections. Both treatment groups had significantly fewer treated pigs compared to the control group (see Table 1 and FIG. 1). The difference in antibiotic requirements between the control group (38.8% of those piglets) and the P120 group (24.4% of those piglets) represents a 37.1% reduction in antibiotic usage. The difference in antibiotic requirements between the control group (38.8% of those piglets) and the P500 group (14.5% of those piglets) represents a 62.6% reduction in antibiotic usage.

Odds ratios were also calculated for the likelihood of antibiotic injections in each treatment group. Piglets in the control group were 3.75 times more likely to receive an antibiotic injection compared to piglets in the P500 group. Similarly, piglets in the control group were 1.97 times more likely to receive antibiotic injections than piglets in the P120 group. Finally, piglets in the P120 group were 1.91 times more likely to receive antibiotic injections than piglets in the P500 group. All of these odds ratio differences were statistically significant at P<0.01. (see Table 2). Piglet Weight Gain: There was no significant difference in piglet birth weight between the groups. However, at weaning, piglets in the P500 group weighed 400 g (0.88 lb) more than piglets in the C group (P<0.05). Piglets in the P120 group weighed 200 g (0.44 lb) more than piglets in the C group (P=0.31) (see Table 3).

Other Piglet Parameters: No significant differences were detected in serum immunoglobulin levels, survival rate or average fecal score between treatment groups.

Sow Parameters

The average lactation period was not significantly different between sows in the treatment groups, ranging from 20.0 to 21.1 days. There were no significant differences between treatment groups in sow weight loss during lactation, dry matter intake or back fat loss.

Conclusions and Discussion: Supplementing sow feed with 3% Px solution in the pre-farrowing and pre-weaning stages reduced antibiotic usage in piglets by a range of 37-62%. Similarly, weaning weights were increased by 200-400 g (0.44-0.88 lb) which is a marked increase in productivity. Both effects followed a dose-response curve, where weaning weight and antibiotic usage showed a larger effect in sows receiving 500 mL Px compared to sows receiving 120 mL Px, and in turn, compared to control sows.

This study was conducted under challenging environmental and health conditions which are encountered in many production operations. The piglet productivity gains and reduction in antibiotic usage were both statistically and practically significant. The economic and veterinary benefits of this reduction in antibiotic usage are highly relevant to todays' challenges in swine production.

P120—Sows given 120 mL/day

P500—Sows given 500 mL/day

TABLE 1
Percentage of Piglets Receiving Antibiotic Injections

| | Control | | P120 | | P500 | |
|---|---|---|---|---|---|---|
| Total number of pigs in group | 415 | | 406 | | 387 | |
| Number and % of pigs receiving | 161 | (38.8%) ^{a} | 99 | (24.4%) ^{a b} | 56 | (14.5%) ^{b} |
| antibiotic injections | | | | | | |

^{a, b} Different superscripts within rows denote significant differences between treatment groups at P <.01

TABLE 2
Odds of Receiving Antibiotic Treatment

| Treatments | Odds Ratio | 95% CI | P value |
|---|---|---|---|
| C vs P500 | 3.75 | 2.653-5.291 | <.01 |
| C vs P120 | 1.97 | 1.455-2.654 | <.01 |
| P120 vs P500 | 1.91 | 1.326-2.739 | <.01 |

TABLE 3
Pig Weights and Average Daily Gain^{†}

| | Control | P120 | P500 |
|---|---|---|---|
| Birth Weight (kg) | 1.4 ± 0.04 | 1.4 ± 0.04 | 1.4 ± 0.04 |
| Weaning Weight (kg) | 4.4 ± 0.15 ^{a} (95% CI 4.1-4.7) | 4.6 ± 0.15 kg ^{a b} (95% CI 4.3- 4.9) | 4.8 ± 0.15 ^{b} (95% CI 4.5-5.1) |
| ADG (g/day) | 148.4 ± 7^{a} | 162.7 ± 7 ^{a b} | 172.5 ± 7 ^{b} |

^{†} Least squares means
^{a b} Different superscripts within rows denote significant differences between treatment groups (P <.05)

1. A method for nourishing a mammalian offspring, characterized by per os administering a pregnant mammal an effective dose of formulation comprising at least two members of a group consisting of steviol glycoside, citric acid monohydrate, monosodium glutamate and glycine; along the period time ranging from third trimester of pregnancy to weaning.

2. A method for increasing the weaning weight of a mammalian offspring, characterized by per os administering a pregnant mammal an effective dose of formulation comprising at least two members of a group consisting of steviol glycoside, citric acid monohydrate, monosodium glutamate and glycine; along the period time ranging from third trimester of pregnancy to weaning.

3. A method for increasing immunoglobulin levels in blood of a mammalian offspring, characterized by per os administering a pregnant mammal an effective dose of formulation comprising at least two members of a group consisting of steviol glycoside, citric acid monohydrate, monosodium glutamate and glycine; along the period time ranging from third trimester of pregnancy to weaning.

4. A method for reducing number of mammalian offspring treated with antibiotics, characterized by per os administering a pregnant mammal an effective dose of formulation comprising at least two members of a group consisting of steviol glycoside, citric acid monohydrate, monosodium glutamate and glycine; along the period time ranging from thirds trimester of pregnancy to weaning.

5. A method for reducing dose of antibiotics required to treat a mammalian offspring up to age of weaning, characterized by per os administering a pregnant mammal an effective dose of formulation comprising at least two members of a group consisting of steviol glycoside, citric acid monohydrate, monosodium glutamate and glycine; along the period time ranging from third trimester of pregnancy to weaning.

6. A method for increasing average daily gain of a mammalian offspring, characterized by per os administering a pregnant mammal an effective dose of formulation comprising at least two members of a group consisting of steviol glycoside, citric acid monohydrate, monosodium glutamate and glycine; along the period time ranging from third trimester of pregnancy to weaning.

7. Oral isotonic compositions characterized by no residual chlorine taste, useful for nourishing a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

8. Oral isotonic compositions characterized by no residual chlorine taste, useful for nourishing a mammalian offspring by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

9. Isotonic drink characterized by no residual chlorine taste, useful for nourishing a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

10. Isotonic drink characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

11. Apple flavoured horses' isotonic drink characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) an apple-flavouring agent.

12. Apple flavoured horses' isotonic drink characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

13. Isotonic taste enhancer characterized by no residual chlorine taste, useful for nourishing a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

14. Isotonic taste enhancer characterized by no residual chlorine taste, useful for nourishing a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

15. Isotonic apple flavoured horses' taste enhancer characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

16. Isotonic Apple flavoured horses' taste enhancer characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

17. An edible powder characterized by no residual chlorine taste, useful for nourishing a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

18. An edible powder characterized by no residual chlorine taste, useful for nourishing a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

19. Apple flavoured horses' edible powder characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

20. Apple flavoured horses' edible powder characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

21. Edible gel characterized by no residual chlorine taste, useful for nourishing a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

22. Edible gel compositions characterized by no residual chlorine taste, useful for nourishing a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

23. Apple flavoured horses' edible isotonic gel characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

24. Apple flavoured horses' edible isotonic gel characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

25. A solid shear thinning thixotropic edible characterized by no residual chlorine taste, useful for nourishing a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

26. A solid shear thinning thixotropic edible gel characterized by no residual chlorine taste, useful for nourishing a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

27. Apple flavoured horses' solid shear thinning thixotropic edible gel characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

28. Apple flavoured horses' solid shear thinning thixotropic edible gel characterized by no residual chlorine taste, useful for nourishing a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

29. Oral isotonic compositions characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

30. Oral isotonic compositions characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

31. A isotonic drink characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

32. A isotonic drink compositions characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

33. Apple flavoured horses' isotonic drink characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

34. Apple flavoured horses' isotonic drink characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

35. An edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

36. An edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

37. An edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

38. An edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

39. Apple flavoured horses' edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

40. Apple flavoured horses' edible powder characterized by no residual chlorine taste, useful for nourishing a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

41. Oral isotonic compositions characterized by no residual chlorine taste, useful for increasing the weaning weight of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

42. Oral isotonic compositions characterized by no residual chlorine taste, useful for increasing the weaning weight of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

43. Isotonic drink characterized by no residual chlorine taste, useful for increasing the weaning weight of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

44. Isotonic drink characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

45. Apple flavoured horses' isotonic drink characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) an apple-flavouring agent.

46. Apple flavoured horses' isotonic drink characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

47. Isotonic taste enhancer characterized by no residual chlorine taste, useful increasing the weaning weight of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

48. Isotonic taste enhancer characterized by no residual chlorine taste, useful for increasing the weaning weight of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

49. Isotonic apple flavoured horses' taste enhancer characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

50. Isotonic Apple flavoured horses' taste enhancer characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

51. An edible powder characterized by no residual chlorine taste, useful for increasing the weaning weight of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

52. An edible powder characterized by no residual chlorine taste, useful for increasing the weaning weight of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

53. Apple flavoured horses' edible powder characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

54. Apple flavoured horses' edible powder characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

55. Edible gel characterized by no residual chlorine taste, useful for increasing the weaning weight of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

56. Edible gel compositions characterized by no residual chlorine taste, useful for increasing the weaning weight of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

57. Apple flavoured horses' edible isotonic gel characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

58. Apple flavoured horses' edible isotonic gel characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

59. A solid shear thinning thixotropic edible characterized by no residual chlorine taste, useful for increasing the weaning weight of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

60. A solid shear thinning thixotropic edible gel characterized by no residual chlorine taste, useful for increasing the weaning weight of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

61. Apple flavoured horses' solid shear thinning thixotropic edible gel characterized by no residual chlorine taste, useful for increasing the weaning weight of a mammalian offspring administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

62. Apple flavoured horses' solid shear thinning thixotropic edible gel characterized by no residual chlorine taste, useful for increasing the weaning weight of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

63. Oral isotonic compositions characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

64. Oral isotonic compositions characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

65. A isotonic drink characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

66. A isotonic drink compositions characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

67. Apple flavoured horses' isotonic drink characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

68. Apple flavoured horses' isotonic drink characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

69. An edible powder characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

70. An edible powder characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

71. An edible powder characterized by no residual chlorine taste, useful increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

72. An edible powder characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

73. Apple flavoured horses' edible powder characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

74. Apple flavoured horses' edible powder characterized by no residual chlorine taste, useful for increasing the weaning weight of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

75. Oral isotonic compositions characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

76. Oral isotonic compositions characterized by no residual chlorine taste, useful for increasing immunoglobulin levels in blood of a mammalian offspring by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

77. Isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

78. Isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

79. Apple flavoured horses' isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) an apple-flavouring agent.

80. Apple flavoured horses' isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

81. Isotonic taste enhancer characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

82. Isotonic taste enhancer characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

83. Isotonic apple flavoured horses' taste enhancer characterized by increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

84. Isotonic Apple flavoured horses' taste enhancer characterized by increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

85. An edible powder characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

86. An edible powder characterized by no residual chlorine taste, useful increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

87. Apple flavoured horses' edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

88. Apple flavoured horses' edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

89. Edible gel characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

90. Edible gel compositions characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

91. Apple flavoured horses' edible isotonic gel characterized by increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

92. Apple flavoured horses' edible isotonic gel characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

93. A solid shear thinning thixotropic edible characterized by no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

94. A solid shear thinning thixotropic edible gel characterized by no residual chlorine taste, useful for increasing immunoglobulin levels in blood of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

95. Apple flavoured horses' solid shear thinning thixotropic edible gel characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

96. Apple flavoured horses' solid shear thinning thixotropic edible gel characterized by no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

97. Oral isotonic compositions characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

98. Oral isotonic compositions characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

99. A isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

100. A isotonic drink compositions characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

101. Apple flavoured horses' isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

102. Apple flavoured horses' isotonic drink characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

103. An edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

104. An edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

105. An edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

106. An edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

107. Apple flavoured horses' edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

108. Apple flavoured horses' edible powder characterized by no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

109. Oral isotonic compositions characterized by no residual chlorine taste, useful for reducing number of mammal offspring treated with antibiotics by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

110. Oral isotonic compositions characterized by no residual chlorine taste, useful for reducing number of mammal offspring treated with antibiotics by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

111. Isotonic drink characterized by no residual chlorine taste, useful for reducing number of mammal offspring treated with antibiotics by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

112. Isotonic drink characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

113. Apple flavoured horses' isotonic drink characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) an apple-flavouring agent.

114. Apple flavoured horses' isotonic drink characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

115. Isotonic taste enhancer characterized by no residual chlorine taste, useful for reducing number of mammal offspring treated with antibiotics by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

116. Isotonic taste enhancer characterized by no residual chlorine taste, useful for reducing number of mammal offspring treated with antibiotics by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

117. Isotonic apple flavoured horses' taste enhancer characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

118. Isotonic Apple flavoured horses' taste enhancer characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

119. An edible powder characterized by no residual chlorine taste, useful for reducing number of mammal offspring treated with antibiotics by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

120. An edible powder characterized by no residual chlorine taste, useful for reducing number of mammal offspring treated with antibiotics by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

121. Apple flavoured horses' edible powder characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

122. Apple flavoured horses' edible powder characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

123. Edible gel characterized by no residual chlorine taste, useful for reducing number of mammal offspring treated with antibiotics by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

124. Edible gel compositions characterized by no residual chlorine taste, useful for reducing number of mammal offspring treated with antibiotics by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

125. Apple flavoured horses' edible isotonic gel characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

126. Apple flavoured horses' edible isotonic gel characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

127. A solid shear thinning thixotropic edible characterized by no residual chlorine taste, useful for reducing number of mammal offspring treated with antibiotics by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

128. A solid shear thinning thixotropic edible gel characterized by no residual chlorine taste, useful reducing number of mammal offspring treated with antibiotics by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

129. Apple flavoured horses' solid shear thinning thixotropic edible gel characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

130. Apple flavoured horses' solid shear thinning thixotropic edible gel characterized by no residual chlorine taste, useful for reducing number of mammal offspring treated with antibiotics by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

131. Oral isotonic compositions characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

132. Oral isotonic compositions characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

133. A isotonic drink characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

134. A isotonic drink compositions characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

135. Apple flavoured horses' isotonic drink characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

136. Apple flavoured horses' isotonic drink characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

137. An edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

138. An edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

139. An edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

140. An edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

141. Apple flavoured horses' edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

142. Apple flavoured horses' edible powder characterized by no residual chlorine taste, useful for reducing number of mammal offspring treated with antibiotics by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

143. Oral isotonic compositions characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

144. Oral isotonic compositions characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

145. Isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

146. Isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

147. Apple flavoured horses' isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) an apple-flavouring agent.

148. Apple flavoured horses' isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

149. Isotonic taste enhancer characterized by no residual chlorine taste, useful reducing dose of antibiotics required to treat a mammalian offspring up to age of weaning by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

150. Isotonic taste enhancer characterized by no residual chlorine taste, useful reducing dose of antibiotics required to treat a mammalian offspring up to age of weaning by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

151. Isotonic apple flavoured horses' taste enhancer characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

152. Isotonic Apple flavoured horses' taste enhancer characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

153. An edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

154. An edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

155. Apple flavoured horses' edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

156. Apple flavoured horses' edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

157. Edible gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

158. Edible gel compositions characterized by no residual chlorine taste, useful reducing dose of antibiotics required to treat a mammalian offspring up to age of weaning by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

159. Apple flavoured horses' edible isotonic gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

160. Apple flavoured horses' edible isotonic gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

161. A solid shear thinning thixotropic edible characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

162. A solid shear thinning thixotropic edible gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

163. Apple flavoured horses' solid shear thinning thixotropic edible gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

164. Apple flavoured horses' solid shear thinning thixotropic edible gel characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

165. Oral isotonic compositions characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

166. Oral isotonic compositions characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

167. A isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

168. A isotonic drink compositions characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

169. Apple flavoured horses' isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

170. Apple flavoured horses' isotonic drink characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

171. An edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

172. An edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

173. An edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

174. An edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

175. Apple flavoured horses' edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

176. Apple flavoured horses' edible powder characterized by no residual chlorine taste, useful for reducing dose of antibiotics required to treat a mammalian offspring up to age of weaning by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

177. Oral isotonic compositions characterized by no residual chlorine taste, useful for increasing average daily gain of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

178. Oral isotonic compositions characterized by no residual chlorine taste, useful for increasing average daily gain of a mammalian offspring by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

179. Isotonic drink characterized by no residual chlorine taste, useful for increasing average daily gain of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

180. Isotonic drink characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

181. Apple flavoured horses' isotonic drink characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) an apple-flavouring agent.

182. Apple flavoured horses' isotonic drink characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

183. Isotonic taste enhancer characterized by no residual chlorine taste, useful for increasing average daily gain of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

184. Isotonic taste enhancer characterized by no residual chlorine taste, useful for increasing average daily gain of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

185. Isotonic apple flavoured horses' taste enhancer characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

186. Isotonic Apple flavoured horses' taste enhancer characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

187. An edible powder characterized by no residual chlorine taste, useful for increasing average daily gain of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

188. An edible powder characterized by no residual chlorine taste, useful for increasing average daily gain of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

189. Apple flavoured horses' edible powder characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

190. Apple flavoured horses' edible powder characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

191. Edible gel characterized by no residual chlorine taste, useful for increasing average daily gain of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

192. Edible gel compositions characterized by no residual chlorine taste, useful for increasing average daily gain of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

193. Apple flavoured horses' edible isotonic gel characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

194. Apple flavoured horses' edible isotonic gel characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

195. A solid shear thinning thixotropic edible characterized by no residual chlorine taste, useful for increasing average daily gain of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

196. A solid shear thinning thixotropic edible gel characterized by no residual chlorine taste, useful for increasing average daily gain of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

197. Apple flavoured horses' solid shear thinning thixotropic edible gel characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

198. Apple flavoured horses' solid shear thinning thixotropic edible gel characterized by no residual chlorine taste, useful for increasing average daily gain of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) An apple-flavouring agent; and (c) prebiotic.

199. Oral isotonic compositions characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

200. Oral isotonic compositions characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

201. A isotonic drink characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

202. A isotonic drink compositions characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

203. Apple flavoured horses' isotonic drink characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) An apple-flavouring agent.

204. Apple flavoured horses' isotonic drink characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

205. An edible powder characterized by no residual chlorine taste, useful increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

206. An edible powder characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

207. An edible powder characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

208. An edible powder characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

209. Apple flavoured horses' edible powder characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

210. Apple flavoured horses' edible powder characterized by no residual chlorine taste, useful for increasing average daily gain of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

211. Oral isotonic compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

212. Oral isotonic compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

213. Isotonic drink characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

214. Isotonic drink characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

215. Isotonic taste enhancer characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

216. Isotonic taste enhancer characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

217. Isotonic apple flavoured horses' taste enhancer characterized by chicken flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

218. Isotonic Apple flavoured horses' taste enhancer characterized by chicken flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

219. An edible powder characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

220. An edible powder characterized by chicken flavour and no residual chlorine taste, useful increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

221. Edible gel characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

222. Edible gel compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

223. A solid shear thinning thixotropic edible characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

224. A solid shear thinning thixotropic edible gel characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

225. Oral isotonic compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

226. Oral isotonic compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

227. A isotonic drink characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

228. A isotonic drink compositions characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

229. An edible powder characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

230. An edible powder characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

231. An edible powder characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

232. An edible powder characterized by chicken flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

233. Oral isotonic compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

234. Oral isotonic compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

235. Isotonic drink characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

236. Isotonic drink characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

237. Isotonic taste enhancer characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

238. Isotonic taste enhancer characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

239. Isotonic apple flavoured horses' taste enhancer characterized by fish flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

240. Isotonic Apple flavoured horses' taste enhancer characterized by fish flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

241. An edible powder characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

242. An edible powder characterized by fish flavour and no residual chlorine taste, useful increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

243. Edible gel characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

244. Edible gel compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

245. A solid shear thinning thixotropic edible characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

246. A solid shear thinning thixotropic edible gel characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

247. Oral isotonic compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

248. Oral isotonic compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

249. An isotonic drink characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

250. An isotonic drink compositions characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

251. An edible powder characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

252. An edible powder characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

253. An edible powder characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

254. An edible powder characterized by fish flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

255. Oral isotonic compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

256. Oral isotonic compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises comprising (a) at least three members of a group consisting steviol glycoside, citric acid monohydrate, monosodium glutamate, glycine, mixtures and derivatives thereof; and (b) prebiotic any derivatives and mixtures thereof.

257. Isotonic drink characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof; and any derivatives and mixtures thereof.

258. Isotonic drink characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) prebiotic any derivatives and mixtures thereof.

259. Isotonic taste enhancer characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting steviol glycoside, citric acid monohydrate, monosodium glutamate, glycine, mixtures and derivatives thereof.

260. Isotonic taste enhancer characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

261. Isotonic apple flavoured horses' taste enhancer characterized by beef flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; and (b) An apple-flavouring agent.

262. Isotonic Apple flavoured horses' taste enhancer characterized by beef flavour and increasing immunoglobulin levels in blood of a mammalian offspring by administering the same to a pregnant mammal along the period time ranging from third trimester of pregnancy to weaning, said composition comprises (a) steviol glycoside, citric acid monohydrate, monosodium glutamate, glycine, mixtures and derivatives thereof; (b) an apple-flavouring agent; and (c) prebiotic.

263. An edible powder characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

264. An edible powder characterized by beef flavour and no residual chlorine taste, useful increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

265. Edible gel characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

266. Edible gel compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting (a) steviol glycoside, citric acid monohydrate, monosodium glutamate, glycine, mixtures and derivatives thereof; and (b) prebiotic any derivatives and mixtures thereof.

267. A solid shear thinning thixotropic edible characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises at least three members of a group consisting of steviol glycoside, citric acid monohydrate, monosodium glutamate, glycine, mixtures and derivatives thereof.

268. A solid shear thinning thixotropic edible gel characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) at least three members of a group consisting steviol glycoside, citric acid monohydrate, monosodium glutamate, glycine, mixtures and derivatives thereof; and (b) prebiotic any derivatives and mixtures thereof.

269. Oral isotonic compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

270. Oral isotonic compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

271. A isotonic drink characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

272. A isotonic drink compositions characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

273. An edible powder characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

274. An edible powder characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

275. An edible powder characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

276. An edible powder characterized by beef flavour and no residual chlorine taste, useful for increasing immunoglobulin levels in blood of a mammalian offspring by administering the same to a pregnant mammal along the period time ranging from third trimester of pregnancy to weaning, said composition comprises (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.
